(19) United States
(12) Patent Application Publication
(10) Pub. No.: US 2016/0354225 A1
(43) Pub. Date: Dec. 8, 2016

(54) CROSS-JOINT-SEGMENTAL ELASTIC ASSISTIVE DEVICE

(71) Applicant: Flyingidea Co., Ltd, Taipei City (TW)

(72) Inventor: Yu-Hung Pan, Taipei City (TW)

(21) Appl. No.: 15/170,745

(22) Filed: Jun. 1, 2016

(30) Foreign Application Priority Data
Jun. 3, 2015 (TW) 104208787

(51) Int. Cl. A61F 5/01

(52) U.S. Cl. CPC A61F 5/0118; A61F 5/0104; A61F 5/0109; A61F 5/0111

(57) ABSTRACT

A cross-joint-segmental elastic assistive device comprises: at least a pair of mutually isolated and opposite separators, such separators respectively having a shield face and a contact face, an elastic connector extending along an elastic force direction and having at least an elastic active section and fixed connection sections respectively located on the both sides of the elastic active section, with each of the fixed connection sections individually being connectedly fixed to one of the separators; and a plurality of fixers connectedly fixing the fixed connection sections of the elastic connector respectively to the separators.

### FIELD OF THE INVENTION

A cross-joint-segmental elastic assistive device for installations on two different joint segments of a user is disclosed.

### BACKGROUND OF THE INVENTION

In daily lives, no matter for athletes or workers required to perform long-termed and repeated actions, it is quite common to undesirably encounter so-called athletic or professional injuries in an extended duration of time, in which the comparably vulnerable locations may exist around people's ankles, knees, elbows, wrists, neck or waist, and many of such areas are close to the joints attached with various body tissues like ligaments, tendons and muscles or the like. Many muscles are across the two sides of a joint and the tendons in the two ends of the muscle respectively bond to the long bones on the both sides of the joint in order to pull the long bones when the muscle contracts or relaxes, such that the joint can bend in conjunction with such muscular contractions or relaxations while the join segments on the two sides of the joint may correspondingly change the spanned angle. In case of physical injuries, every movement of human body may affect the wounded parts thus possibly causing unbearable pain, so the patients generally need to wear certain elastic assistive devices to protect such injured portions and take auxiliary treatment. At the same time, some professional athletes or people who are concerned about possible recurrences of their previous wounds due to heavy workloads or exercises may also put on such elastic assistive devices beforehand thereby preventing this type of injuries.

By applying such patches or assistive devices, it can indeed effectively relieve pain, ameliorate blood circulations and rectify body postures etc., and currently available patches or assistive devices are mostly designed in a structure of entire flat sheet or slice, as shown in FIG. 1. However, if the target location happens to be the joint which usually moves most frequently, the entire patch may easy fall off or shift due to different contraction amounts of the joint or muscles at adjacent portions of the injury. In particular, because the elasticity on the entire patch 9 is the same, suppose the part attached to the joint is subject to intense pull forces, other parts of the patch adhered to the muscles comparatively farther from the joint may not share such deformations, thus resulting in poor effects.

On the other hand, although many assistive devices of hard materials may rely on a fixation structure configured at the two ends thereof to firmly wrap around the two different joint segments, the joint part between these two joint segments can be covered merely by a hard and solid protection without any flexible assistance, nor can this effectively prevent or treat the above-said injuries. Furthermore, this type of hard-material protection may also further restrict the movement of limbs, thus undesirably limiting the activities of athletes or users.

Therefore, in order to resolve the aforementioned issues, the present invention attempts to provide a cross-joint-segmental elastic assistive device which uses at least a pair of mutually isolated separators to be respectively fixed onto joint segments across the two sides a certain joint and allows the elastic connector located there between to be respectively combined to the above-said separators by means of the fixed connection sections on its two sides thus forming an elastic active section between the fixed connection sections such that the elastic active section can successfully cross over the joint and provide sufficient elastic assistive effects. Through this, in addition to muscular force balances nearby the joint, it is also possible to enhance the strength of muscles thereby restoring or maintain normal muscular forces and activity of the joint.

### SUMMARY OF THE INVENTION

An objective of the present invention is to provide a cross-joint-segmental elastic assistive device, which applies an elastic connector to connect mutually isolated and opposite separators, and since the separators are attached merely to the joint segment parts of the user, the joint of the user is not restricted at all thus allowing the user to move freely without limitations.

Another objective of the present invention is to provide a cross-joint-segmental elastic assistive device, in which, the elastic connector is located between different joint segments and provides an elastic force along the orientation of these joint segments, so it is possible to effectively guide and assist the muscular actions thus facilitating better coordinative capabilities of muscles.

Yet another objective of the present invention is to provide a cross-joint-segmental elastic assistive device, in which, since the cross-joint-segmental elastic assistive device is not of an entire plate or slice, the separators are all fixed to the joint segment portions having smaller extensibility rather than attached to the joint part featuring more intense retractions, thus that the cross-joint-segmental elastic assistive device may not easily become loose or shift in use and action.

Hence, in order to achieve the objectives as previously set forth, the present invention provides a cross-joint-segmental elastic assistive device comprising at least a pair of mutually isolated and opposite separators, such separators respectively having a shield face and a contact face, with each of the separators respectively facing towards the user by means of the contact face so as to be detachably fixed to the different joint segments of the user; an elastic connector extending along an elastic force direction; and a plurality of fixers connectedly fixing the fixed connection sections of the elastic connector respectively to the separators.

Moreover, the aforementioned cross-joint-segmental elastic assistive device is characterized in that the elastic connector connecting the separators has at least an elastic active section, and fixed connection sections respectively located on the both sides of the elastic active section, with each of the fixed connection sections individually being connectedly fixed to one of the separators, and, additionally, the elastic connector may be selectively made of an elastic rubber slice, elastic fiber cloth or other suitable materials such that the cross-joint-segmental elastic assistive device disclosed in the present invention allows not only high flexibility in use and comprehensive application ranges, but also can coordinate and control user's postures or actions thereby maintaining the stability of tensions in the muscular group.

### BRIEF DESCRIPTION OF THE DRAWINGS

FIG. 1 shows a side view of a conventional patch applied at the elbow joint of a user, illustrating that, with the same elasticity on the entire patch, the patch may easily shift or exhibit poor adhesion results due to frequent activities around the joint;

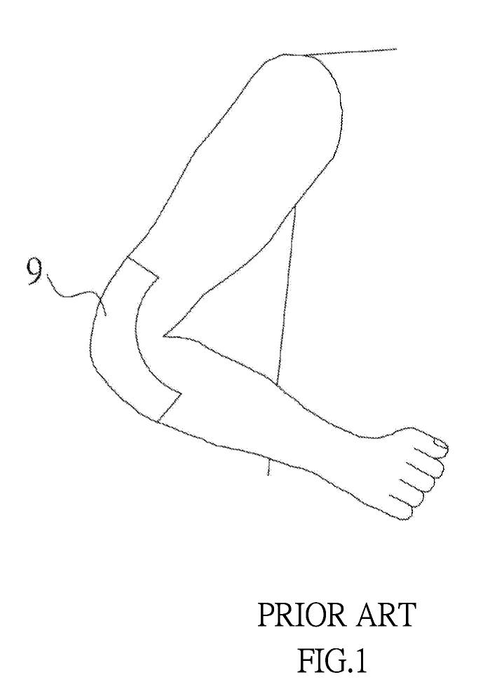

FIG. 2 shows a front view of a preferred embodiment for the cross-joint-segmental elastic assistive device according to the present invention, illustrating the connection relationship between the separators and the elastic connector as well as the structure of the elastic connector;

FIG. 3 shows a side view of the embodiment in FIG. 2, illustrating the up-down correspondence between the separators and the elastic connector;

FIG. 4 shows a side view of the first preferred embodiment for the cross-joint-segmental elastic assistive device according to the present invention utilized at the elbow joint of a user, illustrating the application of the cross-joint-segmental elastic assistive device by the user;

FIG. 5 shows a front view of a second preferred embodiment for the cross-joint-segmental elastic assistive device according to the present invention, illustrating the configurations of the opening and the warning strip on the elastic connector;

FIG. 6 shows a side view of the embodiment in FIG. 5, illustrating the up-down correspondence between the separators and the elastic connector;

FIG. 7 shows a side view of the second preferred embodiment for the cross-joint-segmental elastic assistive device according to the present invention utilized at the elbow joint of a user, illustrating the application of the cross-joint-segmental elastic assistive device by the user; and

FIG. 8 shows a front view of a third preferred embodiment for the cross-joint-segmental elastic assistive device according to the present invention, illustrating the connection between the separators and the elastic connector.

### DETAILED DESCRIPTION OF THE ILLUSTRATED EMBODIMENTS

The aforementioned and other technical contents, aspects and effects in relation with the present invention can be clearly appreciated through the detailed descriptions concerning the preferred embodiments of the present invention in conjunction with the appended drawings; moreover, in each embodiment, the same components will be denoted with similar numbers.

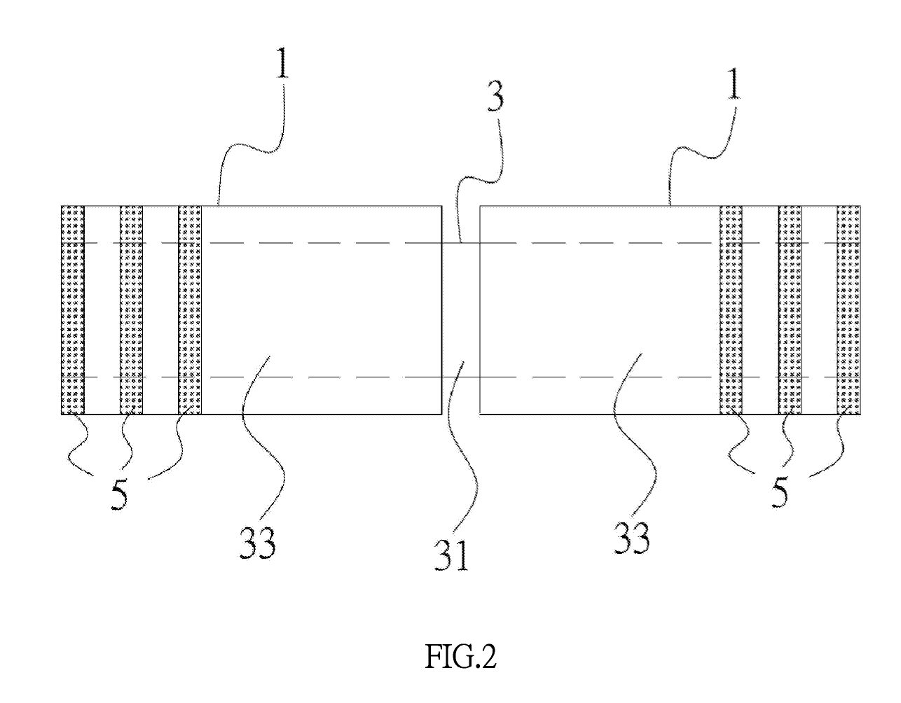

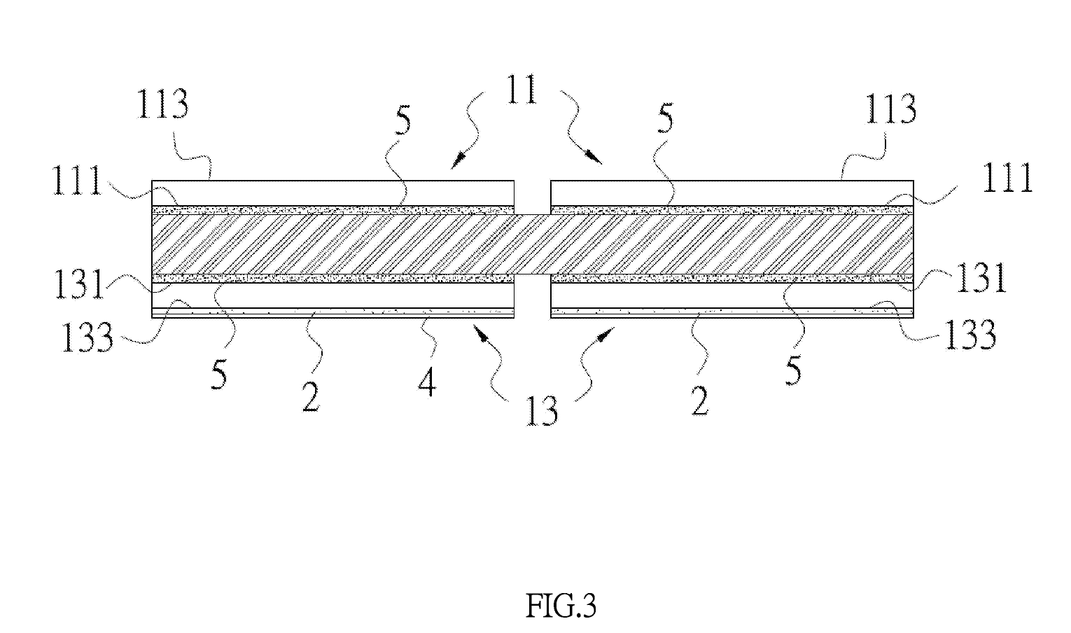

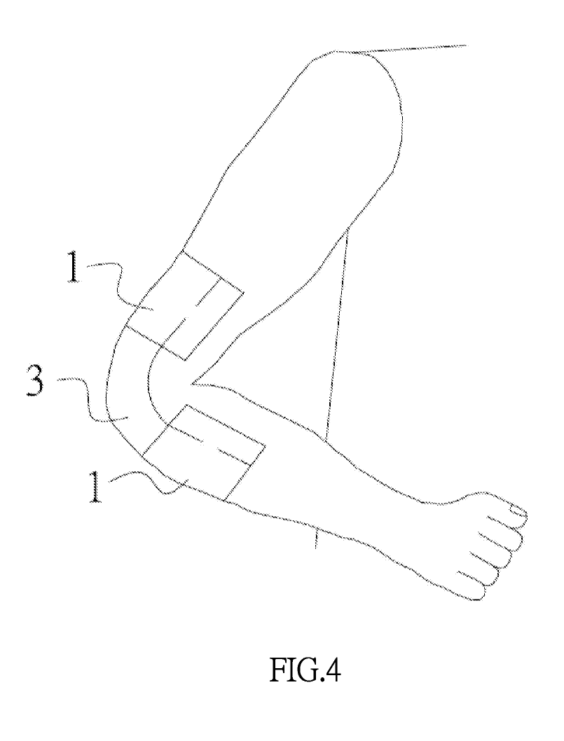

A first preferred embodiment for the cross-joint-segmental elastic assistive device according to the present invention can be respectively referred to FIGS. 2 to 4, in which the present cross-joint-segmental elastic assistive device comprises a pair of mutually isolated and opposite separators 1, as well as an elastic connector 3 installed between such separators 1 for conjunctive movements of such separators 1. To facilitating better understanding, in the present embodiment the separator 1 is exemplified as a slightly elastic patch divided into an upper part and a lower part, in which the lower side adhered to the body surface of the user is referred as the contact face 13, while the upper side opposite to the contact face 13 referred as the shield face 11. In addition, the aforementioned shield face 11 is further divided into an inner side of the shield face 111 facing towards the contact face 13 and an outer side of the shield face 113 exposed to the outside, and the above-said contact face 13 is also further divided into an inner side of the contact face 131 facing towards the contact face 13 and an outer side of the contact face 133 for adhesion.

In the present embodiment, the elastic connector 3 is exemplified as a section of elastic rubber slice and the extension orientation of the elastic rubber slice is denoted as the elastic force direction. Herein the two ends of the elastic connector 3 are respectively snapped in position between the inner side of the contact face 131 and the inner side of the shield face 111 and combined to the separators 1 by means of adhesion, for example. To facilitate better appreciation, the portions on the two ends of the elastic connector 3 for adhesion combination are referred as the fixed connection sections 33, and the glue used to bond the fixed connection patch to the elastic rubber slice is the fixer 5; meanwhile, the portions located between the two fixed connection sections 33 and extending along the elastic force direction without being held in fixation are referred as the elastic active sections 31.

Also, in the present embodiment, each outer side of the contact face 133 facing towards the user is applied with a coating of adhesion layer 2 as well as a release film 4 preventing the adhesion layer 2 from bonding to other unwanted positions. In use of the present product, it needs only to remove the release film 4 from the outer side of the contact face 133 to stick a separator 1 onto the joint segment on the outer side of the triceps muscle in the user's upper arm, then attach the other separator 1 onto the joint segment of the extensor muscle on the outer side of the ulna in the user's lower arm, and make the elastic active section 31 be located at the outer side of the elbow joint. As the elbow joint bends, since the two separators 1 are respectively and firmly adhered to the upper arm and the lower arm, the elastic active section 31 located there between can exert an elastic restoration force upon the elbow joint bending with the elasticity coefficient and elastic force better than each of the separators 1 so as to reduce the risk of excessive force application on elbow joint bending.

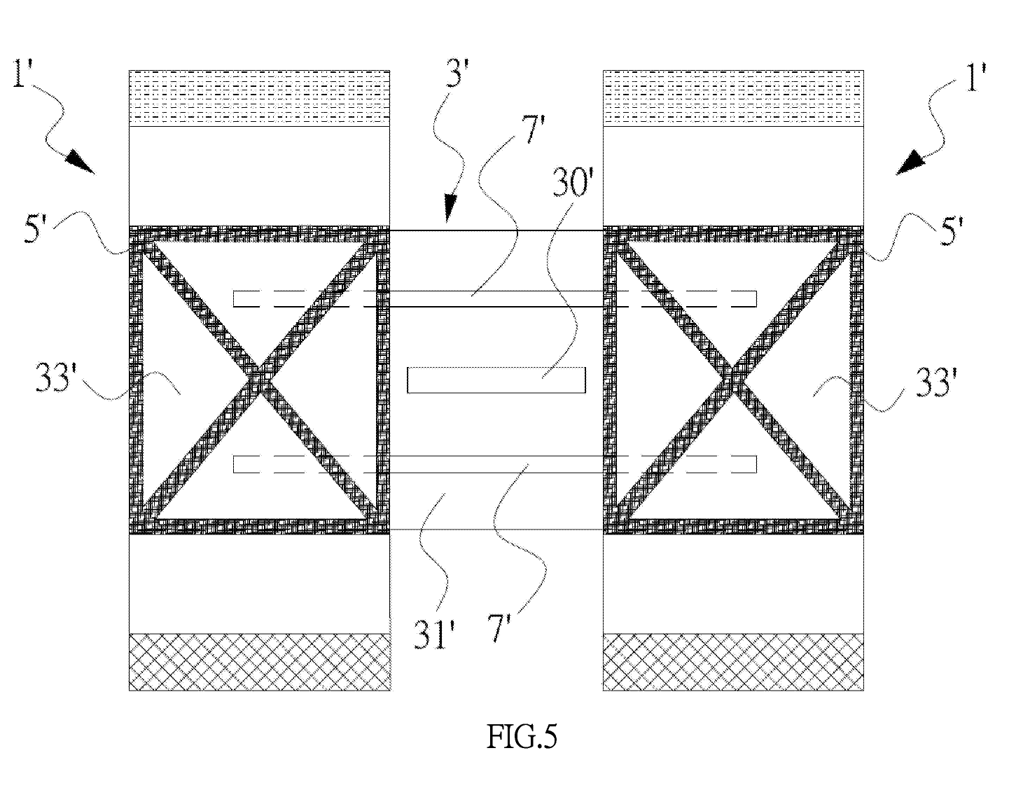

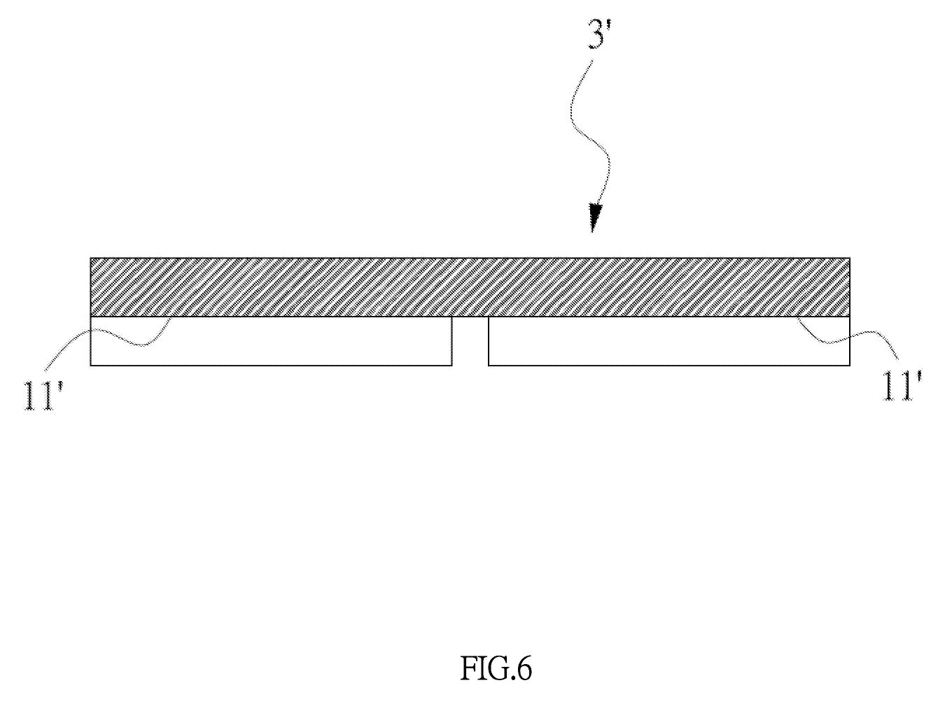

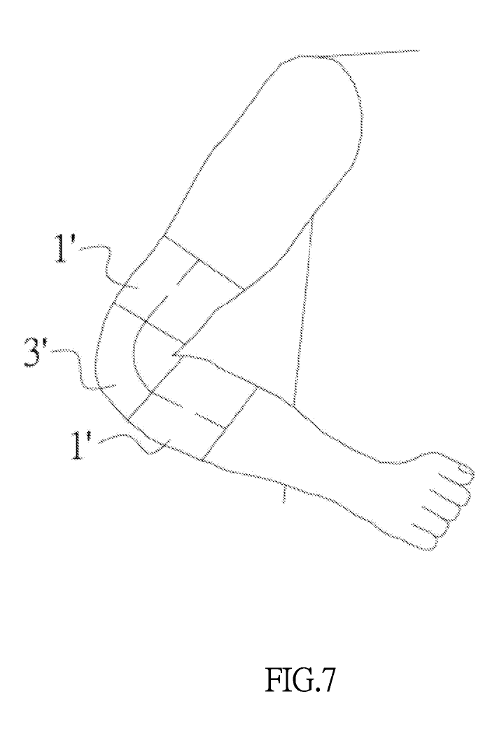

Of course, those skilled ones in the art can conveniently appreciate that the material used in the elastic connector according to the present invention may be selectively other similar materials, such as the elastic fiber cloth, etc. A second preferred embodiment of the present invention is shown in FIGS. 5 to 7, wherein, in the present embodiment, the separators 1′ are exemplified as two long bar-shaped adhesion strips, with the two ends of the long bar-shaped adhesion strips respectively having an adhering part and an adhered part such that the user can detachably fix them to different joint segments for repeated usage. Moreover, in the present embodiment, the fixed connection sections 33′ on the two sides of the elastic connector 3′ are sewn onto the shield face 11′ of the two separators 1′ by using sewing threads as the fixer 5′. Furthermore, in the present embodiment, an opening 30′ is additionally formed within the elastic active section 31′ along the elastic force direction of the elastic connector 3′ in order to correspond to the elbow joint of the user thereby preventing the elastic connector 3′ from deviating.

Especially, due to repeated use of the elastic connector 3′, the elastic fatigue issue may occur in the elastic connector 3′ under long-termed applications; therefore, in the present embodiment, at least a warning strip 7 is additionally woven in the elastic fiber cloth of the elastic connector 3′ along the elastic force direction, which warning strip 7′ having a stretch threshold such that an irrevocable change of color may appear if the deformation exceeds the stretch threshold. Naturally, it is obvious that the elasticity coefficient of the warning strip 7′ should be smaller than the ones in other parts of the elastic connector 3′ so as to remind the user beforehand of potential failures in the product and stop using it.

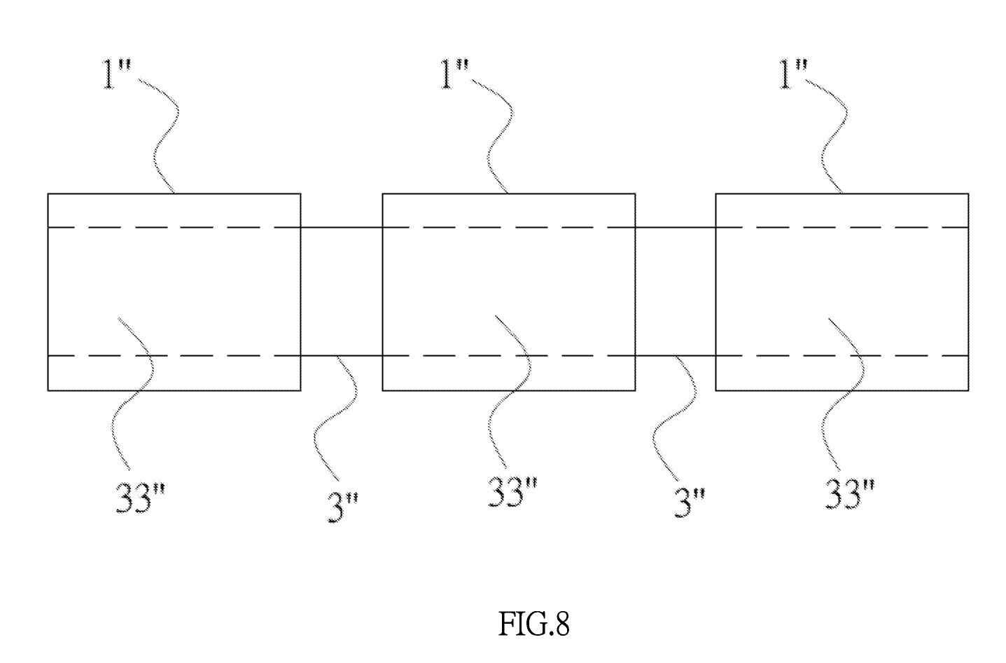

On the other hand, the cross-joint-segmental elastic assistive device according to the present invention is by no means limited to cross over a single joint; for example, to treat an injury on the calf of a patient, it may require to install the elastic assistive device from the thigh, going down through the knee joint, the calf, across the ankle joint and then to the foot. As such, a third preferred embodiment of the present invention is shown in FIG. 8, wherein an elastic connector 3″ extending along the elastic force direction is adopted, and three fixed connection sections 33″ are respectively combined to three separators 1″ in a heat-melted fashion, for example, with each of the separators 1″ being exemplified as an elastic bandage thus allowing to wrap up the contact face towards the skin of the user for installation.

In summary, the present invention utilizes the separators to detachably install the entire cross-joint-segmental elastic assistive device onto different joint segments of a user, and allows the elastic active section in the elastic connector to freely extend and retract so as to exhibit a better auxiliary guiding effect thereby preventing occurrence or deterioration of the injuries; especially, the present invention features a simple integral structure, convenient usage, adaptability to various needs and comprehensive application ranges, and also the structure of the elastic connector enables excellent muscular direction guidance and facilitates enhanced muscular coordination abilities. Moreover, the cross-joint-segmental elastic assistive device according to the present invention can be flexibly used based on the required application positions of the user's body so that the user can move more conveniently, which advances beyond the currently available technologies thus completely achieving the objectives of the present invention. It should be noticed that, however, the illustrations set forth as above simply describe the preferred embodiments of the present invention which are not to be construed as restrictions for the scope of the present invention; on the contrary, all effectively equivalent changes and modifications conveniently made in accordance with the claims and specifications disclosed in the present invention are deemed to be encompassed by the scope of the present invention delineated in the following claims.

What is claimed is:

1. A cross-joint-segmental elastic assistive device for installations on two different joint segments of a user, comprising:
at least a pair of mutually isolated and opposite separators, such separators respectively having a shield face and a contact face, with each of the separators respectively facing towards the user by means of the contact face so as to be detachably fixed to the different joint segments of the user;
an elastic connector extending along an elastic force direction and having at least an elastic active section, and fixed connection sections respectively located on the both sides of the elastic active section, with each of the fixed connection sections individually being connectedly fixed to one of the separators; and
a plurality of fixers connectedly fixing the fixed connection sections of the elastic connector respectively to the separators.

2. The cross-joint-segmental elastic assistive device according to claim 1, wherein the fixers are the adhesion layer applied between the shield face and the contact face.

3. The cross-joint-segmental elastic assistive device according to claim 1, wherein the fixers include the slit line penetrating through the separators and the fixed connection sections.

4. The cross-joint-segmental elastic assistive device according to claim 1, wherein the elastic connector includes at least an elastic rubber slice.

5. The cross-joint-segmental elastic assistive device according to claim 1, wherein the elastic connector includes at least an elastic fiber cloth.

6. The cross-joint-segmental elastic assistive device according to claim 1, wherein the elastic connector further includes a warning strip installed along the elastic force direction.

7. The cross-joint-segmental elastic assistive device according to claim 6, wherein the warning strip has a stretch threshold and an irreversible color change state may occur in case the deformation exceeds the stretch threshold.

8. The cross-joint-segmental elastic assistive device according to claim 7, wherein the warning strip has an elasticity coefficient less than the ones in other parts of the elastic connector.
